(19) United States
(12) Patent Application Publication
(10) Pub. No.: US 2022/0280023 A1
(43) Pub. Date: Sep. 8, 2022

(54) ENDOSCOPE WITH REMOVABLE ELEVATOR

(71) Applicant: UNIVERSITY OF FLORIDA RESEARCH FOUNDATION, INCORPORATED, GAINESVILLE, FL (US)

(72) Inventor: Faisal BUKEIRAT, Jacksonville, FL (US)

(21) Appl. No.: 17/687,346

(22) Filed: Mar. 4, 2022

Related U.S. Application Data
(60) Provisional application No. 63/156,459, filed on Mar. 4, 2021.

(51) Int. Cl. A61B 1/005; A61B 1/018; A61B 1/008

(52) U.S. Cl. CPC A61B 1/0057; A61B 1/0055; A61B 1/0052; A61B 1/018; A61B 1/008

(57) ABSTRACT

An apparatus (100) having: a flexible tube (104) with a proximal end (106), a distal end (108), a lateral aperture (110) in the flexible tube at the distal end, and a guide channel (112) connecting the proximal end to the lateral aperture; an elevator (102) in the distal end having a redirecting surface (130) configured to bend and guide an instrument through the lateral aperture, where movement of the redirecting surface changes a magnitude of a bend in the instrument; and an elevator joint (132) between the elevator and the distal end that permits the movement of the redirecting surface. The elevator joint is configured to release the elevator through the lateral aperture.

### FIELD OF THE INVENTION

The invention relates to endoscopes having a removable elevator.

### BACKGROUND OF THE INVENTION

Endoscopes are devices that can be inserted into the human body and which enable an operator to examine interior surfaces of the human body. Certain endoscopes, for example an endoscopic retrograde cholangiopancreatography (ERCP) duodenoscope, include a camera, a light, and a passageway through the scope through which a tool can be inserted. At a distal end, an elevator can be adjustably positioned and is used to guide the inserted tool as the tool exits the distal end of the endoscope. After a procedure the endoscope is cleaned and disinfected. However, the elevator has been found to be difficult to properly clean and disinfect. This increases infection risk during subsequent use. Accordingly, there is room for improvement in the art.

### BRIEF DESCRIPTION OF THE DRAWINGS

The invention is explained in the following description in view of the drawings that show:

FIG. 1 shows an endoscope with an example embodiment of the elevator disclosed herein.

FIG. 2 is a right side view of the elevator of FIG. 1.

FIG. 3 is a top view of the elevator of FIG. 1.

FIG. 4 is a left side view of the elevator of FIG. 1.

FIG. 5 is a bottom view of the elevator of FIG. 1.

FIG. 6 is a right side view of another example embodiment of the elevator.

FIG. 7 is a left side view of another example embodiment of the elevator.

### DETAILED DESCRIPTION OF THE INVENTION

The present Inventor has devised a unique and innovative solution for the problems associated with cleaning and disinfecting endoscopes with an elevator. Specifically, the Inventor has provided an elevator that is releasably installed at the distal end of the endoscope. This elevator can simply be detached after a procedure. This provides access for cleaning and disinfecting that is not available with the prior art non-detachable elevators. Once the endoscope is cleaned and disinfected, a new elevator can be installed for a subsequent procedure or the original elevator can be reinstalled once cleaned and disinfected. In particular, this elevator can be removed from and installed on its mount while a remainder of the endoscope is in a fully assembled condition. This eliminates any need to remove any portion of the distal end prior to removal of the elevator from its mount.

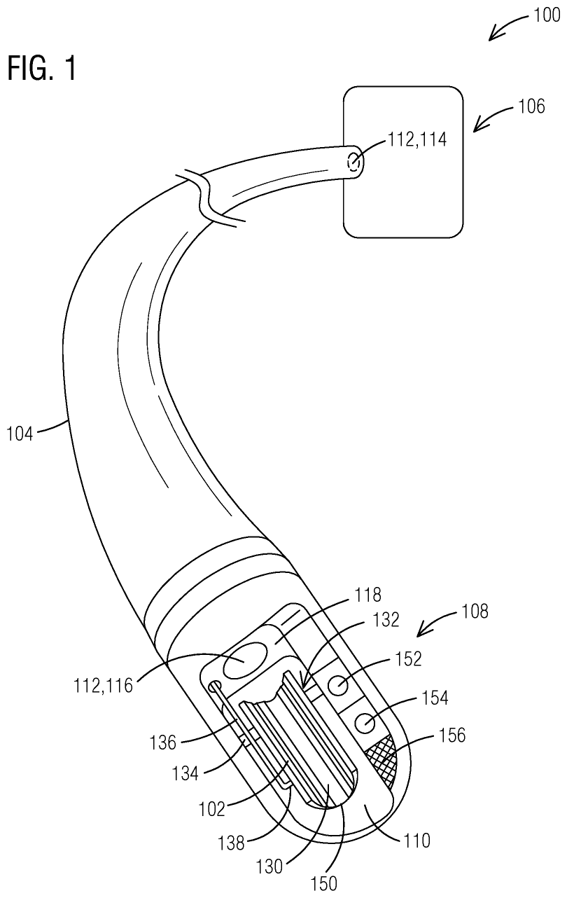

FIG. 1 shows an endoscope 100 with an example embodiment of the elevator 102 disclosed herein. The endoscope includes a flexible tube 104 having a proximal end 106, a distal end 108, a lateral aperture 110 in the flexible tube 104 at the distal end 108, and a guide channel 112 connecting the proximal end 106 to the lateral aperture 110. The guide channel 112 includes a guide channel inlet 114 and a guide channel opening 116 through a bulkhead 118 and adjacent the elevator 102. The elevator 102 comprises a redirecting surface 130 configured to bend and guide an instrument through the lateral aperture 110. Movement of the redirecting surface 130 changes a magnitude of a bend in the instrument. An elevator joint 132 between the elevator 102 and the distal end 108 permits the movement of the redirecting surface 130. The elevator joint 132 is configured to release the elevator 102 through the lateral aperture 110 while the endoscope 100 is otherwise in a fully assembled condition. In this example embodiment, the elevator joint 132 includes a pivot pin 134 onto which the elevator 102 can be mounted and about with the elevator 102 pivots when mounted thereon. In other words, the elevator 102 can be installed and removed through the lateral aperture 110 without having to remove the distal end 108 first. The endoscope further includes an actuating wire 136 configured to move the elevator 102 about the pivot pin 134 and an actuating wire joint 138 configured to releasably connect the actuating wire 136 to the elevator 102.

During operation, the actuating wire 136 pulls on the elevator 102. The actuating wire 136 is disposed above the pivot pin 134 (above is out of the page as seen in FIG. 1). Hence, when the actuating wire 136 pulls on the elevator 102, the elevator 102 pivots/rotates about the pivot pin 134. This lifts a distal end 150 of the elevator 102, and the associated redirecting surface 130 (out of the page as seen in FIG. 1). A tool disposed in the guide channel 112 and in contact with the redirecting surface 130 will be bent toward/through the lateral aperture 110 (out of the page as seen in FIG. 1). A degree/magnitude of the bend will change in response to movement of the redirecting surface 130. In this manner, the tool disposed in the guide channel 112 can be directed in accord with the operator's needs.

The elevator 102 is disposed adjacent an optional air outlet 152, an optional camera 154, and an optional light 156. The endoscope 100 may include additional features such as controls 158 at the proximal end 106.

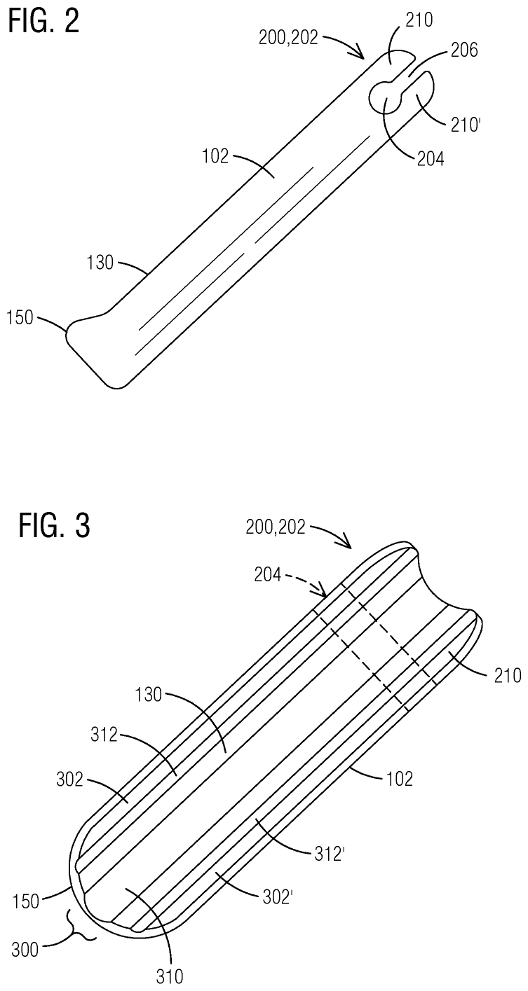

FIG. 2 is a right side view of the elevator 102 with redirecting surface 130 and distal end 150. In this example embodiment, a base end 200 of the elevator 102 includes a snap connector 202 configured to permit the elevator 102 to be installed onto and removed from the pivot pin 134 (see FIG. 1) through the lateral aperture 110 (see FIG. 1. The snap connector 202 comprises a recess 204, a slot 206 leading to the recess 204, and at least one prong 210, 210′. In this example embodiment, the recess 204 includes a cylindrical shape. This way the recess 204 is configured to receive and rotate about the pivot pin 134 (FIG. 1). The prongs 210, 210′ are configured to flex to permit the elevator 102 to be pressed onto or pulled off of the pivot pin 134 and to retain the pivot pin 134 (FIG. 1) when the pivot pin 134 is in the recess 204. In this example embodiment, there are two prongs 210, 210′ and the recess 204 is disposed between the two prongs 210, 210′. However, there may be only one prong or more than two prongs in certain embodiments.

FIG. 3 is a top view of the elevator 102 of FIG. 1 with redirecting surface 130 and distal end 150. The redirecting surface 130 is optionally curved such that a middle region 300 is recessed (into the page as seen in FIG. 3) between sides 302, 302′ so that the redirecting surface 130 can cradle an inserted tool that is being guided thereby. An optional central groove 310 may be present whether or not the redirecting surface 130 is concave. Similarly, optional side grooves 312, 312′ may be present whether or not the redirecting surface 130 is concave.

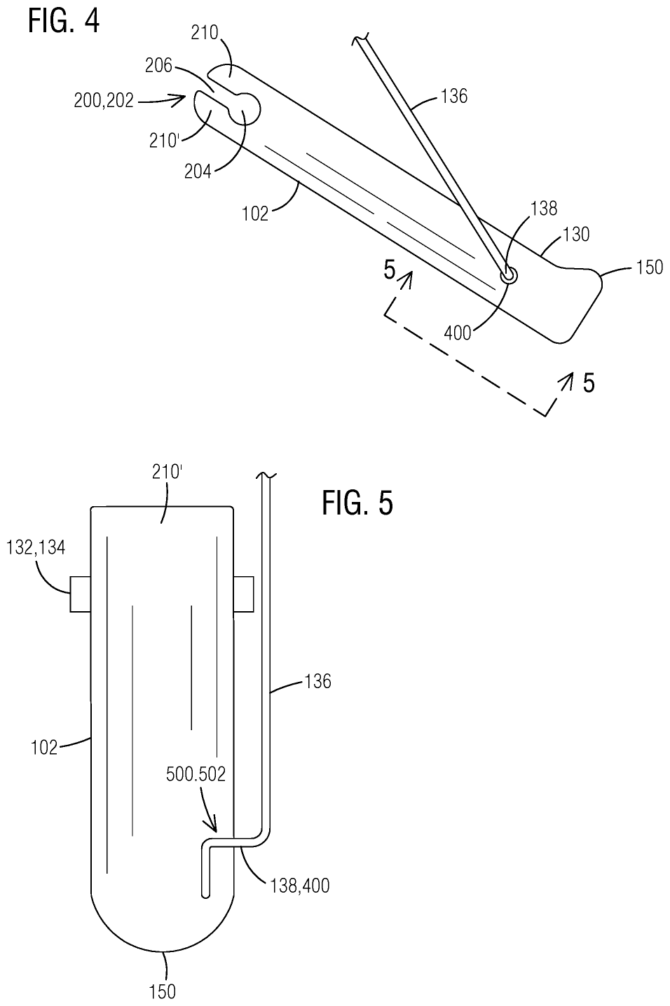

FIG. 4 is a left side view of the elevator 102 of FIG. 1. The actuating wire 136 is connected to the elevator 102 at the actuating wire joint 138. The actuating wire joint 138 includes a hole 400 in the elevator 102. The other elements shown in FIG. 4 are the same as that of FIG. 2.

FIG. 5 is a bottom view of the elevator 102 along line 5-5 in FIG. 4. The actuating wire joint 138 comprises a z-shaped connection 500 at a distal end 502 of the actuating wire 136. The actuating wire 136 can be made slack so that the elevator 102 can be manually pulled from the pivot pin 134 using fingers or a hand tool such as pliers. Once the elevator 102 is free from the pivot pin 134, the elevator can be worked off the z-shaped connection 500 at a distal end 502 of the actuating wire 136. The freed elevator 102 can be disposed of. Alternately, the freed elevator 102 can be cleaned and disinfected. The endoscope 100 can be properly cleaned and disinfected because there is sufficient access to do so now that the elevator 102 has been removed. Once the endoscope 100 has been properly cleaned and disinfected, a new elevator 102 or a properly cleaned and disinfected elevator 102 can be installed on the pivot pin 134 so that the endoscope 100 will be ready for a subsequent procedure.

The Artisan will readily understand that other suitable ways of attaching the actuating wire 136 to the elevator 102 are acceptable.

The elevator 102 may be composed of a material that is suitably resilient that the prongs 210, 210′ will retain the elevator 102 on the pivot pin 134 during normal operation, but which will release the elevator 102 upon the application of external manual force. In an embodiment, the elevator 102 comprises plastic at least for the prongs 210, 210′. In an embodiment, the elevator is also galvanized. This resists adherence of any particular matter or debris including bacteria to the elevator 102.

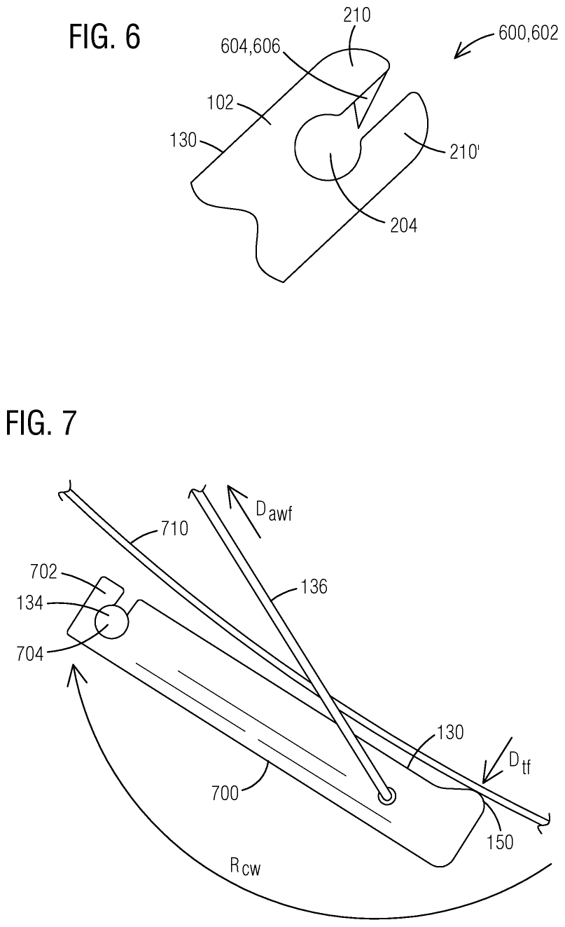

FIG. 6 is a right side view of another example embodiment of the elevator 600 with a snap connector 602 having a retention feature 604. In this example embodiment, the retention feature 604 is a barb 606. The barb 606 will assist in retaining the pivot pin 134 in the recess 204. The retention feature 604 may optionally be sacrificial. A sacrificial retention feature 604 will yield to permit installation of the elevator 600 onto the pivot pin 134. However, the geometry of the retention feature 604 will prevent removal of the elevator 600 from the pivot pin 134 without breaking the retention feature 604. This will ensure that the elevator 600 does not prematurely release from the pivot pin 134. The retention feature 604 can also be configured to ensure that a new elevator 600 is used as a replacement when an elevator 600 with a broken retention feature 604 will not properly mount to the pivot pin 134. For example, when the retention feature 604 is a barb 606, if the barb 606 is broken, the elevator 102 will not hook onto the pivot pin 134. An installer would immediately recognize this because the elevator 600 with the broken barb would simply not engage the pivot pin 134. Moreover, the retention feature 604 would be configured to block access to the recess 204 when broken. The other features shown in FIG. 6 are provided in FIGS. 2-4.

FIG. 7 is a left side view of another example embodiment of the elevator 700. In this example embodiment, there is a single prong 702 and it is oriented at ninety (90) degrees to the prongs 210, 210′ of the embodiment of FIG. 2. This configuration helps ensure the pivot pin 134 remains in the recess 704. During operation, a tool 710 is shown in contact with the distal end 150 of the elevator 700. A resilient of the tool 710 resists the bending and imparts a tool force in a direction Dtf on the distal end 150 of the elevator 700. The actuating wire 136 pulls on the elevator 700 to effect the bending of the tool. This generates an actuating wire force acting in a direction Dawf. These vectors urge the elevator 700 to rotate clockwise in direction Rcw. This, in turn, urges the recess 704 onto the pivot pin 134, thereby increasing the securement of the elevator 700 in place during operation. While prongs in two configurations have been shown, the Artisan will understand other configurations are possible depending on specifics of the endoscope 100 and the operating conditions/environment.

Although not depicted, it is possible for the elevator to have a cylindrical shape, where the redirecting surface is disposed in a side of the elevator, and where a base end of the elevator is connected to the pivot pin 134 via a threaded connection. In such an embodiment, a tip end of the elevator may include a tool feature configured to receive a tool that turns the elevator and thereby threads the threaded connection. Such an embodiment would not work with the z-shaped connection 500 at the distal end 502 of the actuating wire 136. This is because the elevator would need to be installed onto the threaded connection first before the wire is connected to the elevator. Once, so installed, the elevator could not be worked onto the z-shaped connection 500. However, other actuating wire joints could be used with the cylindrically shaped elevator.

While various embodiments of the present invention have been shown and described herein, it will be obvious that such embodiments are provided by way of example only. Numerous variations, swapping of features among embodiments, changes, and substitutions may be made without departing from the invention herein. Accordingly, it is intended that the invention be limited only by the spirit and scope of the appended claims.

The invention claimed is:

1. An apparatus comprising:
a flexible tube comprising a proximal end, a distal end, a lateral aperture in the flexible tube at the distal end, and a guide channel connecting the proximal end to the lateral aperture;
an elevator in the distal end comprising a redirecting surface configured to bend and guide an instrument through the lateral aperture, wherein movement of the redirecting surface changes a magnitude of a bend in the instrument; and
an elevator joint between the elevator and the distal end that permits the movement of the redirecting surface;
wherein the elevator joint is configured to release the elevator through the lateral aperture.

2. The apparatus of claim 1, wherein the elevator joint comprises a pin about which the elevator rotates, and wherein the elevator comprises a snap connector configured to permit the elevator to be installed onto and removed from the pin through the lateral aperture.

3. The apparatus of claim 2, wherein the snap connector comprises a recess and at least one prong, wherein the recess is configured to receive and rotate about the pin, and wherein the at least one prong is configured to flex to permit the elevator to be pressed onto or pulled off of the pin and to retain the pin when the pin is in the recess.

4. The apparatus of claim 3, wherein the at least one prong comprises two prongs, and wherein the recess is disposed between the two prongs.

5. The apparatus of claim 1, wherein the elevator joint comprises a pin about which the elevator rotates, and wherein the elevator comprises a sacrificial connector configured to break when the elevator is pulled through the lateral aperture.

6. The apparatus of claim 2, wherein when bending the instrument the elevator experiences an urge to rotate in a first direction, and wherein the urge to rotate presses the pin into the snap connector.

7. The apparatus of claim 1, wherein the elevator releases without removal of a portion of the distal end comprising the elevator.

8. The apparatus of claim 1, further comprising:
an actuating wire configured to move the elevator about the elevator joint; and
an actuating wire joint configured to releasably connect the actuating wire to the elevator.

9. The apparatus of claim 8, wherein the actuating wire joint comprises a z-shaped connection at a distal end of the actuating wire and a through-hole in the elevator configured to receive the z-shaped connection.

10. A method comprising
Obtaining an apparatus of claim 1,
Using the apparatus for an endoscope procedure, and
Removing the elevator from the endoscope at the elevator joint.

11. The method of claim 10, further comprising re-attaching the elevator to the endoscope.

12. The method of claim 10, further comprising attaching a different elevator to the endoscope.

13. The method of claim 2, further comprising removing the elevator from the endoscope at the elevator joint, wherein removing the elevator comprises removing the elevator from the elevator pin.

14. The method of claim 13, further comprising reattaching the elevator to the elevator pin.

15. The method of claim 13, further comprising attaching a different elevator to the elevator pin.

16. The method of claim 13, wherein the elevator comprises a retention feature.

17. The method of claim 16, wherein the retention feature comprises a barb.

18. The method of claim 16, wherein the retention feature breaks upon removal thereby disallowing reattaching of the elevator to the elevator pin.
